(19) United States
(12) Patent Application Publication
(10) Pub. No.: US 2008/0102081 A1
(43) Pub. Date: May 1, 2008

(54) Preparations and applications for treatment of high cholesterol levels

(76) Inventors: Brian Alexis, Torrance, CA (US); Emiliya Kosturkova, Sofia (BG)

Correspondence Address: Cislo & Thomas LLP, 1333 2nd Street, Suite #500, Santa Monica, CA, 90401-4110, US

(21) Appl. No.: 11/500,842

(22) Filed: Aug. 8, 2006

(51) Int. Cl. A61K 36/06; A61P 3/06; A61P 35/00

(52) U.S. Cl. 424/195.16

(57) ABSTRACT

A novel composition of modified strains of Monascus spp. which produce biologically active products useful as therapeutic treatments is disclosed. The novel composition of modified Monascus strains disclosed has a high level of total antioxidant activity and is useful as therapeutic composition, such as for lowering serum cholesterol and triglycerides in mammals, for the treatment of tumors and for as a therapeutic agent in the treatment of Alzheimer's and Parkinson's disease. Also disclosed are methods for producing novel biologically active products from Monascus strains and novel Monascus mixtures that yield products with the desired biological activities.

### FIELD OF THE INVENTION

This invention relates generally to composition and methods of their use involving fungi of the genus Monascus, to a novel composition of a mixture of strains of Monascus spp. which produce biologically active substances useful in therapeutic treatments. The novel composition of strains disclosed herein possesses high level of total antioxidant activity and is useful as a therapeutic composition, such as for lowering serum cholesterol and triglycerides, for the treatment of tumors and for as a therapeutic agent in the treatment of Alzheimer's and Parkinson's disease.

### BACKGROUND

Heart disease is cited as the number one killer of women and men in the United States. Each year, more than a million Americans have heart attacks, and about a half million people die from heart disease. High blood cholesterol and an imbalance of cholesterol and triglycerides are major risk factors in heart disease. Cholesterol is a common component of mammalian cell membranes and functions to help stabilize the membranes. It is also an important precursor for the biosynthesis of other steroids (bile acids) and essential hormones. Thus, cholesterol is a crucial molecule in animals and humans, although high levels in the blood may contribute to atherosclerosis.

Most of the body's supply of cholesterol is synthesized in the liver where the amount of cholesterol produced is controlled by the enzyme HMG-CoA (Hydroxymethyl-glutaryl CoA) reductase, which catalyzes the reduction of HMG-CoA to mevalonate. When more cholesterol is needed, more of the enzyme is produced in liver cells to increase cholesterol production. Alternatively, when there is enough cholesterol in the body, less of the enzyme is produced to decrease cholesterol production.

Conventional first-line treatment of high cholesterol levels most often includes the use of a group of compounds known as statins, which inhibit HMG-CoA reductase and thus the reduction of HMG-CoA to mevalonate. However, there exist data that as many as 40% of patients with established coronary heart disease and hypercholesterolaemia do not achieve target plasma concentrations of cholesterols (see EUROASPIRE I and II Group; European Action on Secondary Prevention by Intervention to Reduce Events. Clinical reality of coronary prevention guidelines: A comparison of EUROASPIRE I and II in nine countries. Lancet, 2001, 357(9261), 995-1001). The inadequate treatment is likely to be caused by a combination of insufficient pharmacological effect and a reluctance to perform the necessary multiple statin dose escalations, in part due to concern for potentially serious adverse effects such as muscle and liver toxicity (see, Stein, E. A. An investigative look: Selective cholesterol absorption inhibitors—embarking on a new standard of care. American Journal of Management Care, 2002, 8(2 Suppl.), S36-S39).

The serious adverse effects, as well as milder adverse effects such as gastrointestinal discomfort and insomnia, after use of statins are all dose related (see, Clausen, P., Lindhardsen J., Høie, L., Stender, S. Treatment with AbacorR, a soy-based dietary supplement, further reduces plasma concentrations of total and low-density lipoprotein cholesterol in statin-treated hypercholesterolaemic patients. Innovative Food Science and Emerging Technologies, 2004, 5 377-383).

In addition, while generally well tolerated by most patients, statins have been associated with serious side effects in some individuals, such as rhabdomyolysis, a life-threatening muscle breakdown. For example, Cerivastatin was withdrawn from the U.S. market due to a fatal rhabdomyolysis rate 16 to 80 times higher than other statins based on Federal Food and Drug Administration reports using a denominator of prescription volume (3.16 fatal cases/million prescriptions versus 0.15 for the statin class as a whole) (see, Staffa, J. A., Chang, J., Green L. Cerivastatin and reports of fatal rhabdomyolysis. N. Eng. J. Med., 2002; 346:539-540). Although the actual degree of risk associated with the use of statins is the subject of heated debate, the ability of statins to produce myopathy under some circumstances is well established.

Also, elevated hepatic transaminase generally occurs in 0.5% to 2% of patients on statin therapy and is reported to be dose dependent. See, Hsu, I., Spinler, S. A., Johnson, N. E. Comparative evaluation of the safety and efficacy of HMG-CoA reductase inhibitor monotherapy in the treatment of hypercholesterolemia. Ann. Pharmacotherapy, 1995; 29: 743-59. Recently, an announcement noted possible legal action against the manufacturer of Atorvastatin could be undertaken after it was alleged that liver and kidney damage have been observed in the patients prescribed this HMG-COA reductase inhibitor (see, http:/www.classactionamerica.com/public/caseIndex.aspx?1ngCaseID=1048). Again, in addition to rhabdomyolysis, the results from the clinical trials suggest an elevation of hepatic enzyme activity by 0.7 percent for patients at lower dosages and much higher rate for those taking the highest prescribed (80 mg) dose.

Statins have also been associated with increased risk when used together with other medications, such as cyclosporine, fibrates, azole antifungals, warfarin, nefzodone antidepressant, itraconazole, ketoconazole, macrolide antibiotics, erythromycin and clarithromycin, HIV protease inhibitors, verapamil, and amidoarone. Combinations of statins and these medications must be used with caution or avoided altogether. See, Pasternak et al., Clinical Advisory on the use and safety of statins, JACC Vol. 40, No. 3, 2002:567-72

For example, combining statins with a fibrate is attractive for persons who have both high serum cholesterol and high triglycerides, or for those who continue to have elevated triglycerides after reaching their LDL-cholesterol target on statin therapy. However, there may be concern about an increased danger of developing myopathy with this combination. In addition, statins may increase the risk of myopathy and/or are contraindicated for patients who are over 80 years, frail, on HIV protease inhibitors, in cases of cholestasis and active liver disease, alcohol abuse and for patients who have multisystem disease. (see, e.g., Phillips P. S., Haas, R. H., Bannykh, S. et al., Statin-associated myopathy with normal creatine levels. Ann. Intern. Med., 2002; 137:581-585). Because there is also higher risk for diabetic patients with chronic renal failure and for patients who are continued on statin therapy during hospitalization for major surgery, statin therapy may need to be withheld during such periods.

In addition, it has been found out that HIV-infected patients on HAART (highly active anti-retroviral therapy) protocol—a combination of 1-2 protease inhibitors and 2-3 nucleoside analogs often develop serious lipid metabolism disorder, with elevated cholesterol and triglyceride levels, resulting in disfiguring fat deposits (“buffalo hump” and/or “protease paunch”), increased risk for heart disease, including heart attack, and development of insulin resistance and diabetes—a situation that necessitates the application of cholesterol-lowering drugs. Unfortunately, in patients on HAART, it has been established that statin-based drugs do not always exert their activity (see Henry, K. et al., Atorvastatin and gemfibrozil for protease-inhibitor-related lipid abnormalities. The Lancet, 352, 26 Sep. 1998, 1031-1032).

The use of statins and cancer risk is also a subject of a heating controversy. Several studies have suggested that statins have an inhibitory effect on cancer cell proliferation in vitro, invasion and metastasis in an animal model and in humans, when used for adjuvant treatment (see, Kusama, T. et al., Inhibition of epidermal growth factor-induced RhoA translocation and invasion of human pancreatic cancer cells by 3-hydroxyl-3-methylglutaryl-coenzyme A reductase inhibitors. Cancer Res 2001 61: 4885-91; Kusama, T. et al. 3-hydroxy-3-methylglutaryl-coenzyme A reductase inhibitors reduce human pancreatic cancer cell invasion and metastasis. Gastroenterology 2002 122(2):308-17; Kawata, S. et al., Effect of pravastatin on survival in patients with advanced hepatocellular carcinoma. A randomized controlled trial. Br J Cancer 2001 84: 886-91).

The mechanism by which statins exert their antitumor activity has been associated with the triggering of tumor-specific apoptosis (see, Wong, W. et al., HMG-CoA reductase inhibitors and the malignant cell: the statin family of drugs as triggers of tumor-specific apoptosis. Leukemia 2002, 16: 508-19. In addition, in vitro studies indicate antiproliferative properties of Lovastatin on some retinoic acid-sensitive pediatric cancers and squamous cell carcinomas, but not on breast cancer cells (see, Dimitroulakos, J. et al., Differential Sensitivity of Various Pediatric Cancers and Squamous Cell Carcinomas to Lovastatin-induced Apoptosis: Therapeutic Implications. Clinical Cancer Research 2001, Vol. 7, 158-167).

Other studies, however, show that under experimental conditions, statins have been shown to increase the frequency of several cancers in rodents, at levels of exposure similar to that used therapeutically in humans (see, Newman, T. B. and S. B. Hulley. Carcinogenicity of lipid-lowering drugs. JAMA 1996, 275: 55-60). Recent in vitro studies demonstrate that statins can act as regulators of the immune system, and it is suggested that they could contribute to the development of malignant diseases by this mechanism (see, Kwak, B. et al., Statins as a newly recognized type of immunomodulator. Nat Med 2000, 6: 1399-402; Weitz-Schmidt, G. et al., Statins selectively inhibit leukocyte function antigen-1 by binding to a novel regulatory integrin site. Nat Med 2001, 7: 687-92).

The laboratory findings, however, have not been translated into clinical benefit. Some studies present evidence of an increase of the incidence of cancer after prolonged application of statins to elderly individuals (see, pages at URLhttp://www.thincs.org/unpublic.UR3.htm) and some meta-analyses of randomized clinical trials have suggested that cholesterol-lowering drugs may increase noncardiovasular mortality (see, Newman, T. B. and S. B. Hulley. Carcinogenicity of lipid-lowering drugs. JAMA 1996, 275: 55-60). Other studies (see, e.g., Dale, K. et al., Statins and Cancer Risk A Meta-analysis. JAMA 2006, 295:74-80) find null effect on cancer incidence. However, the average duration of the trials was 5 years, but most patients taking stains would expect to do so for life. The fact that cancers can occur after long latency periods following exposure to the carcinogen shows that we do not yet have the length of follow-up studies necessary to exclude a carcinogenic effect of statins. Thus, millions of asymptomatic people are being treated with medications, the ultimate effects of which are not yet known.

Hence, the development of an effective non-statin composition for use in the treatment of abnormal lipid metabolism, including high cholesterol levels and for the treatment of statin-resistant cancers would therefore be a significant benefit and would be an attractive alternative to the use of statins.

### SUMMARY

The instant invention relates to compositions and methods for regulating cholesterol levels in a patient with therapeutic compositions that are statin-free. The compositions disclosed herein are derived from a combination of modified Monascus strains and can be used as therapy to lower serum cholesterol in humans without the use of statins. The composition and methods disclosed herein are useful in treating conditions such as cardiovascular diseases, high blood cholesterol, cholesterol plaques, to improve arterial and vein blood circulation and condition of the blood vessel walls, to support detoxification functions of the liver, to restore liver metabolism without increasing enzyme production, to act as an anti-tumor agent and to reduce the degenerative process in Alzheimer's and Parkinson's disease.

Fungi, belonging to the genus Monascus have been used in the food industry, such as for example, in fermenting red rice and red rice wine, as food coloring agents and as preservatives for meat and fish. Certain Monascus strains, such as Monascus purpureus, Monascus anka, Monascus major, and Monascus rubiginosus have also been employed in the food industry and cosmetic industries due to their ability to synthesize red, yellow, and orange pigments.

The new compositions disclosed herein comprise the fermentate of a novel combination of Monascus anka, Monascus kaoliang, and Monascus vini and lower cholesterol levels, total lipids and triglycerides without side effects and have a wide range of applications. The fermentate, and compositions comprising the fermentate, may also be referred to herein as “the Monascus compositions.” These new Monascus compositions disclosed herein contains no Lovastatin (active substance mevinolin) and its use, therefore, avoids the side effects and risks of the statin-based therapies. In addition, the Monascus compositions disclosed herein have extremely low toxicity in high dosages; have no observed antibiotic effects and are cytotoxic to human mammary carcinoma cells.

The methods and Monascus compositions disclosed herein, including the mode of administration and preparation process, increase effectiveness at low dosages and maximize total antioxidant and other bioactivity, thereby supporting liver metabolism of cholesterol without increasing production of enzymes in the liver and assist in restoring liver function without side effects. In one aspect of the invention, a novel composition of Monascus spp. named Strain MB 1000 Bulgaricus (“Strain MB 1000 BG”) having a high level of total antioxidant activity has been developed. In another aspect of the invention, the methods and Monascus compositions disclosed herein improve liver metabolism without increasing enzyme production in the liver. The bioactive substances and Monascus compositions disclosed herein may be administered in suitable forms known in the art, such as capsules, tablets, liquid preparations, food supplements and in cosmetic and topical preparations.

The above noted objects and other objects of the invention are preferably accomplished by the use of Strain MB 1000 BG derived from a combination of Monascus anka, strain ZM01 (NBIMCC 8455); Monascus kaoliang, strain ZM02 (NBIMCC 8456) and Monascus vini, strain ZM03 (NBIMCC 8457). These strains were further cultivated as disclosed herein, archived and used in the main fermentation process.

In general, the fermentation process disclosed herein preferably comprises the steps of 1) cultivating the modified Monascus strains on solid media; 2) transferring the Monascus cultures to an intermediate liquid medium; 3) transferring the biomass to fermentation reactors in a fermentation liquid culture medium; and 4) repeated deep feed batch cultivation in main fermentation reactor.

After fermentation, the production process may be completed and the liquid biomass is preferably transferred to a disintegrator. After disintegration, the fermentate is preferably transferred to a spray dryer. From the dryer, the product may be transferred to a packaging department. The fermentation liquid culture medium used in the fermentation process preferably comprises a combination of organic and inorganic carbon and nitrogen sources, and mineral salts. For example, the fermentation liquid culture medium may comprise from about 10% sugars, about 12% organic and inorganic nitrogen sources, about 1% of mineral salts from any organic or inorganic source.

### BRIEF DESCRIPTION OF THE DRAWINGS

FIG. 1 is an HPLC profile of the Lovastatin standard at 62 ppm.

FIG. 2 is an HPLC profile of the Lovastatin standard at 31 ppm.

FIG. 3 is an HPLC profile of the Lovastatin standard at 12.4 ppm.

FIG. 4a is an HPLC profile of injection 1 for sample 2 of the Monascus fermentate for Lovastatin analysis.

FIG. 4b is an HPLC profile of injection 2 for sample 2 of the Monascus fermentate for Lovastatin analysis.

FIG. 5a is an HPLC profile of injection 1 for sample 3 of the Monascus fermentate for Lovastatin analysis.

FIG. 5b is an HPLC profile of injection 2 for sample 3 of the Monascus fermentate for Lovastatin analysis.

FIG. 6 is an HPLC profile of a blank sample of the Monascus fermentate for Lovastatin analysis.

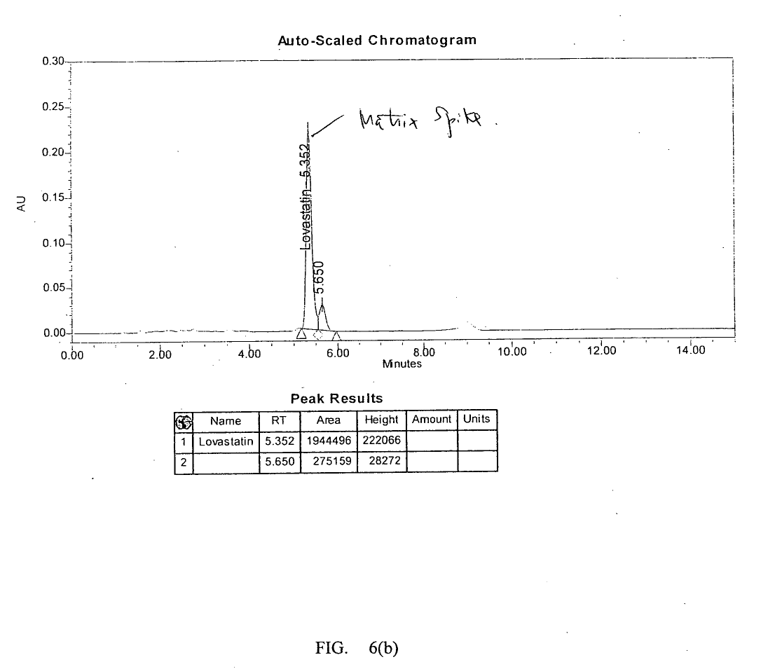

FIG. 7 is a standard curve obtained by using dilutions of 62, 31 and 12.4 ppm of Lovastatin.

FIGS. 8(a)-8(d) show cell cultures from the CHO cell line after 24 hour treatment with Strain MB 1000 BG: 8(a) control culture; 8(b) Strain MB 1000 BG at 10 mg/ml; 8(c) Strain MB 1000 at 20 mg/ml; and 8(d) Strain MB 100 BG at 40 mg/ml.

FIG. 9a-9d show cell cultures from the BALB/c 3T3 cell line after 24 hour treatment Strain MB 1000 BG: 9(a) control culture; 9(b) Strain MB 100 BG at 8 mg/ml; 9(c) Strain MB 1000 at 15 mg/ml; and 9(d) Strain MB 100 BG at 40 mg/ml.

FIGS. 10(a)-10(c) show Monascus composition, Strain MB 1000 BG (aqueous solution) at concentrations 1 and 10 mg/ml on the test microorganism Bacillus subtilis, strain 1709: 10(a) control culture displaying no activity of solvent, 10(b) absence antibacterial activity without daylight exposure, 10(c) absence of photodynamic effect.

FIGS. 11(a)-11(c) show Monascus composition, Strain MB 1000 BG (aqueous solution) at concentrations 1 and 10 mg/ml on the test microorganism Staphylococcus aureus subsp. aureus, strain 509:

11(a) control culture, 11(b) absence antibacterial activity without daylight exposure, 11(c) absence of photodynamic effect.

FIGS. 12(a)-12(b) show cell cultures treated with Monascus composition, Strain MB 1000 BG (aqueous solution) at concentrations 1 and 10 mg/ml on the test microorganism Escherichia coli WF+:

12(a) no activity of solvent, 12(b) absence antibacterial activity without daylight exposure, and 12(c) absence of photodynamic effect.

FIG. 13 shows cell viability (%) plotted against dose (mg/ml) of Monascus composition on MCF-7 cultures as assayed by Neutral Red Uptake Assay.

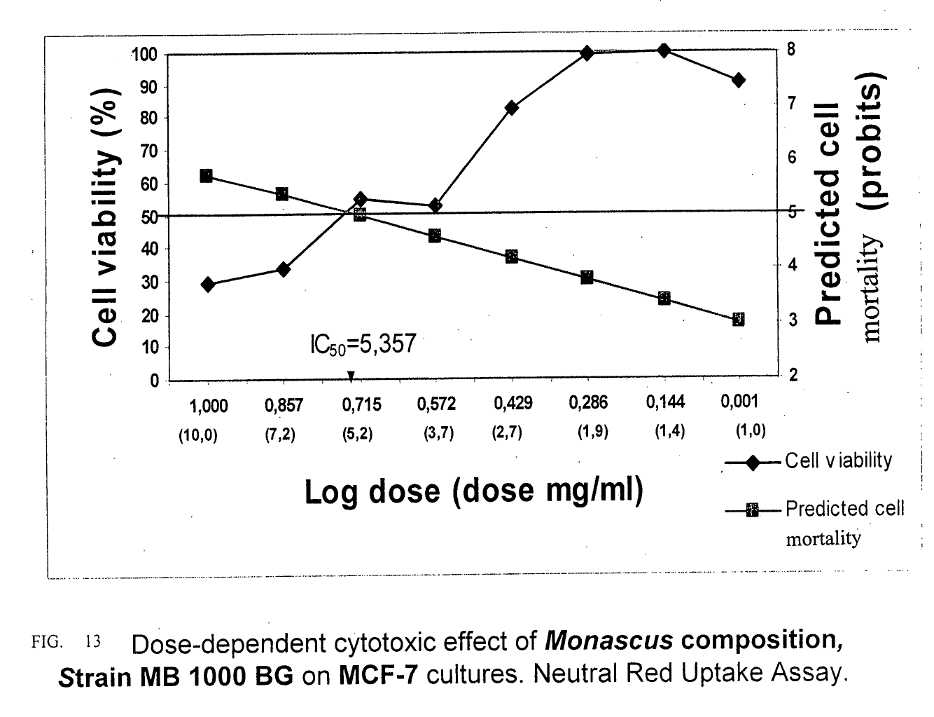

FIG. 14 shows the number of viable cells plotted against dose (mg/ml) of Monascus composition on MCF-7 cultures using the dye-exclusion test.

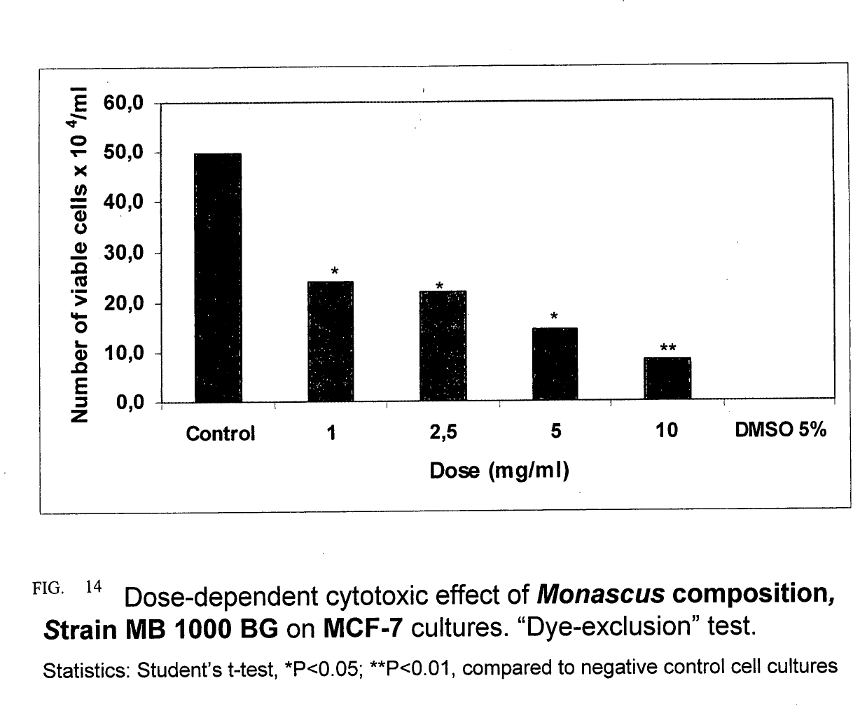

FIG. 15 is a series of micrographs showing cytoxicity of Monascus composition after treating breast cell carcinoma cell line MCF-7 for 24 hours: FIG. 15(a)-negative control culture; FIG. 15(b) positive control cell culture (5% DMSO); FIG. 15(c) Strain MB 1000 BG 1 mg/ml; FIG. 15(d) Strain MB 1000 BG 2.5 mg/ml; FIG. 15(e) Strain MB 1000 BG 5 mg/ml; and FIG. 15(f) Strain MB 1000 BG 10 mg/ml.

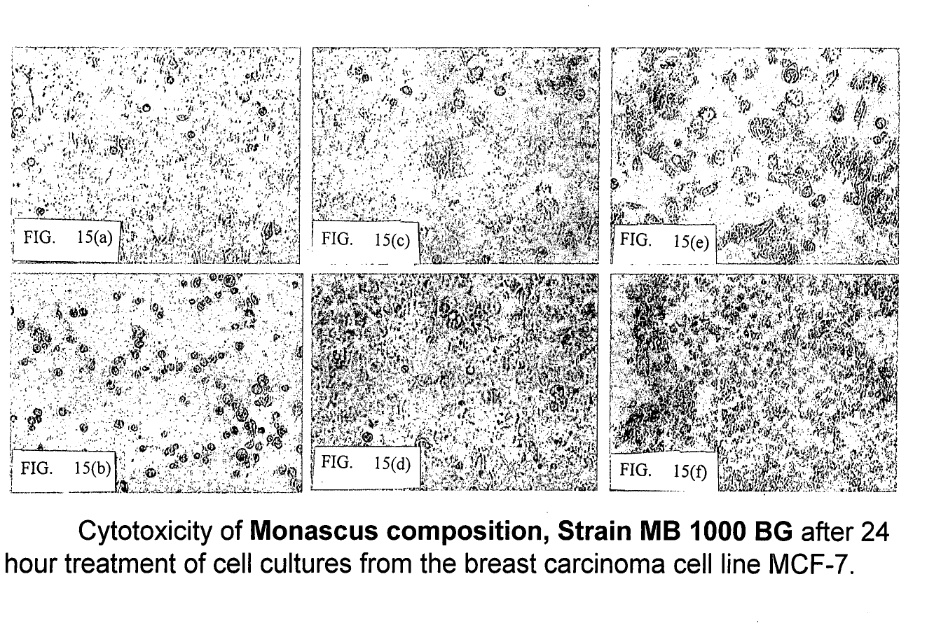

### DETAILED DESCRIPTION

The detailed description set forth below in connection with the appended drawings is intended as a description of exemplary embodiments and is not intended to represent the only forms in which these embodiments may be constructed and/or utilized. The description sets forth the functions and the sequence of steps for constructing and operating the exemplary. embodiments. However, it is to be understood that the same or equivalent functions and sequences may be accomplished by different embodiments that are also intended to be encompassed within the spirit and scope of the specification.

The compositions and methods disclosed herein are preferably derived from fermentation of a novel mixture of Monascus strains and are effective in lowering serum cholesterol without the use of statins. The therapeutic Monascus compositions disclosed herein comprise a novel composition created by fermentation of Strain MB 1000 BG, made from a mixture of Monascus anka Strain ZM01 (NBIMCC 8455); Monascus kaoliang Strain ZM02 (NBIMCC 8456) and Monascus vini Strain ZM03 (NBIMCC 8457), each of which were deposited on Apr. 14, 2006 at the National Bank of Industrial Microorganisms and Cell Cultures (NBIMCC), 1113 Sofia, 125 Tsarigradsko chausse blvd., block 2, Bulgaria, and were issued, respectively, Accession Nos. 8455, 8456, 8457 on Apr. 14, 2006, and are readily available from NBIMCC as indicated by the Accession Nos. given in parentheses.

Monascus Strain MB 1000 BG has the following characteristics:

The Strain MB 1000 BG is capable of synthesizing products that do not contain Lovastatin. The fermentation product from Strain MB 1000 BG displays low toxicity in high dosage. The fermentation product (or fermentate) from Strain MB 1000 BG displays no antibiotic effects. The fermentate from Strain MB 1000 BG contains total antioxidant and other bioactivity improving liver metabolism of cholesterol without increasing production of enzymes in the liver and without other side effects. The fermentate from Strain MB 1000 BG is also cytotoxic to human mammary carcinoma cells.

The liquid culture medium used in the fermentation process preferably contains a combination of organic and inorganic carbon and nitrogen sources, and mineral salts. The liquid culture medium preferably comprises about 10% sugars, such as for example, glucose, maltose, galactose, fructose, glucosamine, galactosamine, alcohols, xylitol, sorbitol, ribitol, glycerol, carbonates, manitol, but may comprise from about 0.0001% to about 20% sugars. The liquid culture medium may additionally comprise preferably about 12% organic and inorganic nitrogen sources, such as, yeast extract, malt extract, malt broth, yeast autholyzate, any starch containing products including rice powder, corn powder, potato powder, polypeptides, peptides, urea, glycopeptides, glycopolypeptides, NaNO₃, NHCl, (NH)₂SO₄, and may comprise from about 0.001% to about 20% of organic and inorganic nitrogen sources. The liquid culture medium may also comprise preferably about 1% of mineral salts and essential elements from organic or inorganic source—CaCl, MgSO₄, Se, Zn, Cu, Ag, AuCl, Mn, and may comprise from about 0.001% to about 5% of mineral salts and essential elements from organic or inorganic sources.

The mixture of Monascus anka, Monascus kaoliang and Monascus vini is preferably combined in a ratio of Monascus anka Strain ZM01 at about 33.3%; Monascus kaoliang Strain ZM02 at about 33.3% and Monascus vini Strain ZM03 at about 33.3%, but may also be combined in a ratio of from about 0.001% to about 99.999% each of Monascus anka, Monascus kaoliang and Monascus vini.

### EXAMPLE 1

### Cultivation of the Modified Monascus Strains

Monascus anka ZM01 strain was cultivated in Petri dishes on sterilized solid media preferably containing (g/l): agar (from about 20 to about 25 g/l), peptone (from about 5 to about 10 g/l), malt extract (from about 5 to about 10 g/l) and glucose (from about 20 to about 25 g/l) at a pH of from about 5 to about 6. The cultivation is carried out at between about 28 degrees C. to about 30 degrees C. for from about 5 to about 10 days. The solid media is preferably sterilized before the cultivation process in an autoclave at approximately 120° C. to approximately 126° C. and at about 1 atm to about 1.1 atm for between about 25 to about 35 minutes.

Monascus kaoliang ZM02 strain was cultivated in vials on sterilized solid media preferably containing (g/l): agar (from about 20 to about 25 g/l), peptone (from about 5 to about 10 g/l), yeast autholizate (from about 5 to about 10 g/l) and glucose (from about 20 to about 25 g/l) at a pH of from about 5 to about 6. The cultivation is preferably carried out at between about 28° C. to about 30° C. for from about 5 to about 10 days. The solid media is preferably sterilized before cultivation in an autoclave at between about 120° C. to about 126° C. for between about twenty five minutes to about thirty five minutes at a pressure of between about 1 atm to about 1.1 atm.

Monascus vini ZM03 strain was cultivated in vials on sterilized solid media preferably containing (g/l): agar (from about 20 to about 25 g/l), peptone (from about 5 to about 10 g/l), yeast autholizate (from about 5 to about 10 g/l) and glucose (from about 20 to about 25 g/l) at a pH of from about 5 to about 6. The cultivation is carried out at between about 28° C. to about 30° C. for from about 5 to about 10 days. The solid media is preferably sterilized before cultivation in an autoclave at from between 120° C. to about 126° C. for from about twenty five minutes to about thirty five minutes at a pressure of from between 1 atm to about 1.1 atm.

### Preparation of Intermediate Liquid Culture Medium and Cultivation in Liquid Medium

After cultivation on solid media for about 5 to about 7 days, Monascus strains ZM01, ZM02 and ZM03 were transferred to a flask containing intermediate liquid culture medium. The intermediate culture medium contained organic and inorganic carbon sources (about 7%); inorganic nitrogen sources (about 1.2%), organic nitrogen sources (about 12%); and mineral salts (about 2%). The intermediate culture medium may also be adjusted as needed to contain from about 0.01% to about 10% of organic and inorganic carbon sources carbon sources, from about 0.01% to about 5% of inorganic nitrogen sources, from about 0.01% to about 15% organic nitrogen sources and from about 0.0001% to about 5% of mineral salts.

Cultivation in the intermediate liquid medium is preferably carried out on a shaker at between about 24° C. to about 39° C. for from about twenty hours to about seventy-two hours at a pH of about 4 to about 6. The intermediate liquid culture medium is preferably sterilized in an autoclave at between about 120° C. to about 126° C. for from about twenty five minutes to about thirty five minutes at a pressure of between about 1 atm to about 1.1 atm.

### Main Fermentation Process

The cultivated mixture of Monascus anka ZM01, Monascus kaoliang ZM02 and Monascus vini ZM03 was transferred from intermediate liquid medium culture to a fermentation liquid culture medium for the main fermentation. The fermentation liquid culture medium comprised organic and inorganic carbon sources (about 10%); inorganic nitrogen sources and organic nitrogen sources (about 12%); and mineral salts (about 1%) at a pH of from about 5 to about 6 and at a temperature of between about 28° C. to about 30° C. and aeration of from about 1.0 liters/min to about 1.3 liters/min and at an agitation of about 500 r.p.m to about 600 r.p.m. The fermentation liquid culture medium may also be adjusted to comprise from about 0.01% to about 15% of organic and inorganic nitrogen sources, from about 0.01% to about 5% of inorganic nitrogen sources, from about 0.01% to about 20% of organic nitrogen sources, and from about 0.0001% to about 5% of mineral salts.

### Feed Batch Cultivation

After between about 30 hours to about 40 hours in the main fermentation reactor, cultivation is completed. Repeated deep feed batch cultivation of the Monascus composition Strain MB 1000 BG was then carried out with a sequence of from 2-8 hours and from 4-10 changes of main fermentation liquid culture medium with fresh fermentation liquid culture medium. The volume of exchanged main fermentation culture medium was about 40% to about 70% each time.

### Disintegration Process

After repeated deep feed batch fermentation, all liquid biomass was transferred to a disintegrator where disintegration of the Monascus cells is started under a pressure of about 500 atm to about 800 atm.

### EXAMPLE 2

### Measurement of Antioxidant Activity

Total antioxidant activity of Monascus composition, Strain MB 1000 BG was tested against a standard of vitamin C by the method of Jork, H., et al., Thin-Layer Chromatography Volume 1A and 1B. VCH, Weinheim, 1989 and 1990. Total antioxidant activity of the Monascus composition, Strain MB 1000 BG was measured from 1,200 to 1,800 OU/mg substance.

### EXAMPLE 3

### Clinical Studies

Volunteers provided data cards containing anamnesis, family anamnesis and history of accompanying disorders, diagnosis, previous treatment and motivation of future treatment with Monascus composition, Strain MB 1000 BG. For each participant, Total Cholesterol (TC), HDL-C, LDL-C, triglycerides (TG) were determined. The participants were monitored for 2-6 weeks. Improvement in TC, TG and LDL-C levels occurred in 4 of the 6 participants, with the percentage decrease after the second week of treatment shown as summarized in Table 1.

TABLE 1
Percentage Decrease After Two Weeks of Treatment with Monascus
composition, Strain MB 1000 BG (daily dosage 65 mg).

| N | TC | TG | LDL-C |
|---|---|---|---|
| 1 | 26.8 | 21.3 | 26.5 |
| 2 | 22.1 | 13.6 | 22.5 |
| 3 | 14 | 13.6 | 18.8 |
| 4 | 16.6 | 17.7 | 22.9 |
| Average | 19.8 | 16.6 | 22.6 |

### EXAMPLE 4

### Analysis of Lovastatin Content

Analysis of Lovastatin content in Monascus composition Strain MB 1000 BG was performed by INC Laboratories, Irvine, Calif. using USP method for Lovastatin. The sample run sequence for analysis of the substance produced by Monascus composition, Strain MB 1000 BG (lot # 0602589, Job ID # 73510 and 73506) is shown in Table 2.

TABLE 2
Sample Run Sequence for Lot #'s 73510 and 73506

| # | Name | Method | Injection Vol. | Run T |
|---|---|---|---|---|
| 1 | Lovastatin std | Lovastatin MS | 20.00 | 15 |
| 2 | 73510 | Lovastatin MS | 20.00 | 15 |
| 3 | 73510 MS | Lovastatin MS | 20.00 | 15 |
| 4 | Lovastatin std. | Lovastatin MS | 20.00 | 15 |
| 5. | Lovastatin std. | Lovastatin MS | 20.00 | 15 |
| 6 | 73506 | Lovastatin MS | 20.00 | 15 |
| 7. | Blank | Lovastatin MS | 20.00 | 15 |
| 8. | Blank | Lovastatin MS | 20.00 | 15 |

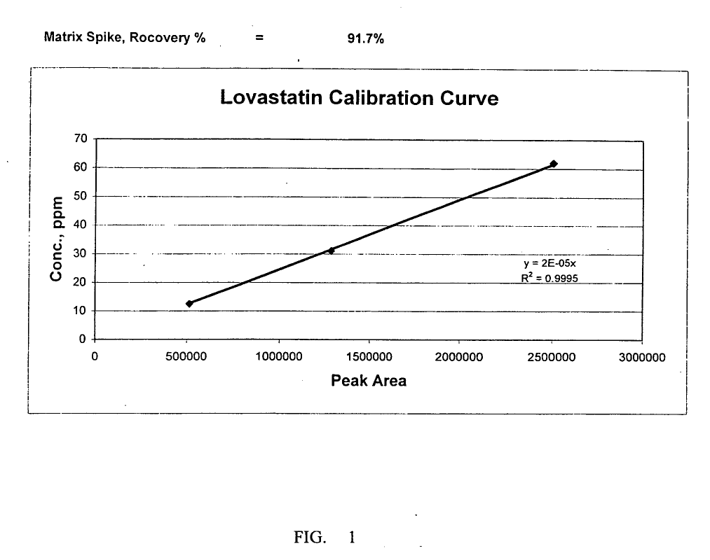

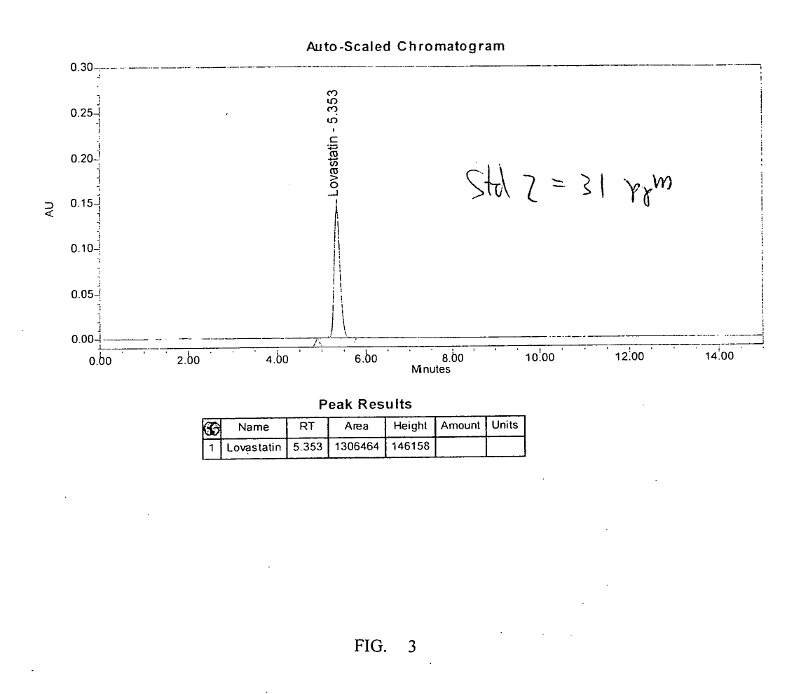

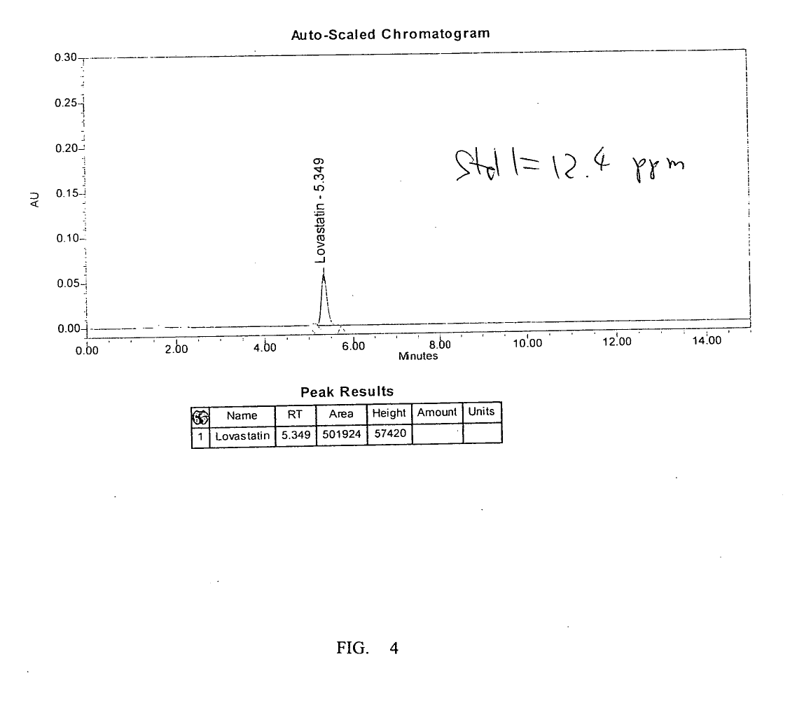

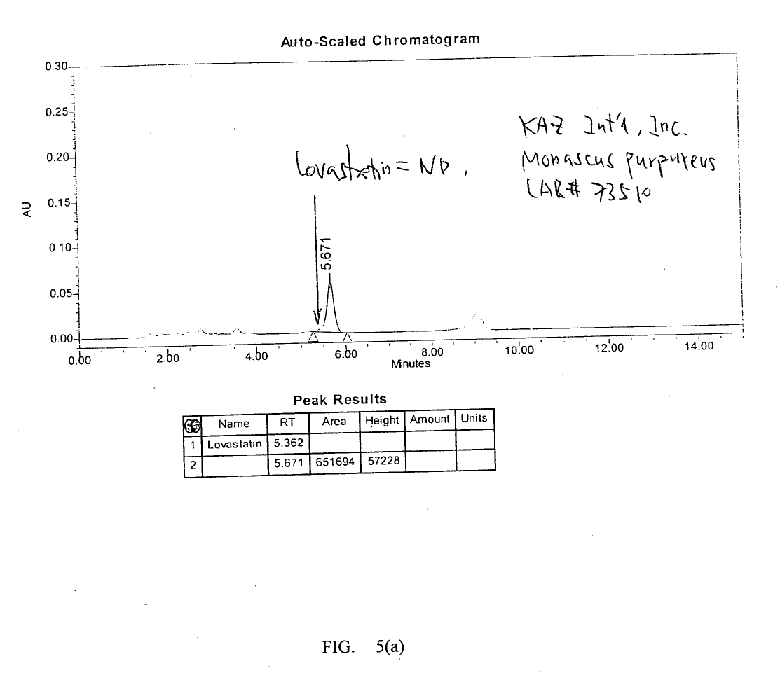

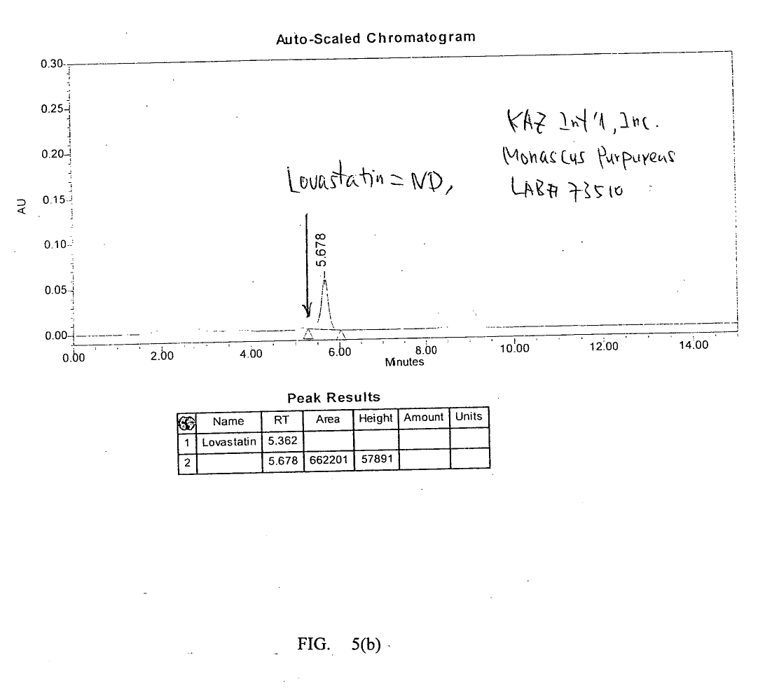

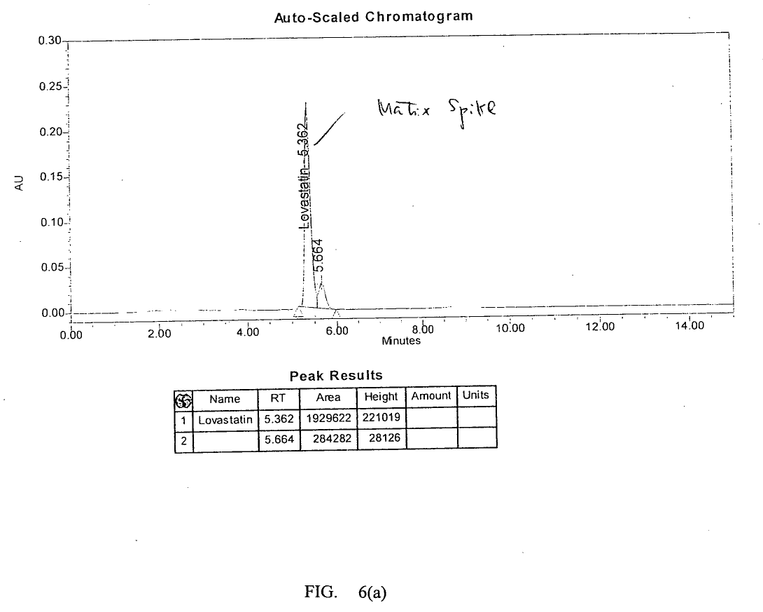

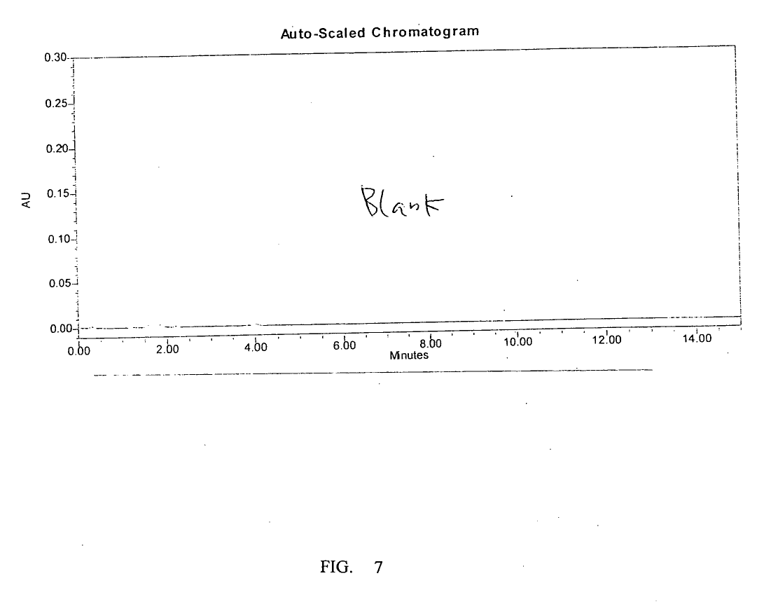

Chromatographic separation was achieved on Agilent Zorbax C8 column. Acetonitrile:H₃PO₄ (65:35 v/v) was used as the mobile phase. The eluent was pumped at a flow-rate of 1.5 ml/min. The UV detector was at 238 nm. The injection volume was 20 μl and the run time was fifteen minutes. Lovastatin standards (sample #'s 1, 4, 5, Lovastatin USP RS lot # H2C012) were run at, respectively, 62 ppm, 31 ppm and 12.4 ppm. Results from running the first, second and third standards at, respectively 62, 31 and 12.4 ppm are shown in FIG. 1-FIG 3. Injections 1 and 2 for sample 2 are shown in FIG. 4a-FIG. 4b. Injections 1 and 2 for sample 3 are shown in FIG. 5a-5b. A standard curve was obtained by using dilutions of 62, 31 and 12.4 ppm of Lovastatin origin in acetonitrile (FIG. 7).

### EXAMPLE 5

### Cytotoxicity of Substance Strain MB 1000 BG

Cytotoxicity of Monascus composition Strain MB 1000 BG was carried out at the Bulgarian Academy of Science, Institute of Experimental Pathology and Parasitology in Sofia, Bulgaria. Cytotoxicity studies were performed using BALB/c 3T3 clone 31 mouse embryo cells (see, Aaronson, S. and G. Todaro. Development of 3T3-like lines from Balb/c mouse embryo culture. J. Cell Physiol., 72, 1968, 141148), obtained from Centro Substrati Cellulari (Brescia, Italy) and Chinese hamster ovary (CHO) continuous cell lines (see, Puck, T. J. Exp. med., 108, 1958, 945) and confirmed that the substance exhibited extremely low toxicity.

BALB/c 3T3 clone 31 and CHO cells were grown as monolayers in 75 cm² tissue culture flasks (Cellstar, Greiner Bio-One GmbH) at 37 degrees centigrade in a humidified atmosphere with 5% CO_{2.} BALB/c 3T3 cells were grown in Dulbecco's Modified Eagle's Medium (DMEM), (Sigma). CHO cells were grown in Nutrient Mixture Ham's F 12 (Sigma). Both cell cultures were supplemented with 5% FBS (BioWhittaker Europe) and penicillin (100 UI/ml) and Streptomycin (100 μg/ml). The cells were routinely passaged at a cell density of ˜1×10⁶ cells in 75 cm² flasks every 3-4 days (average doubling time is 20-24 hours).

The substance from Monascus composition, Strain MB 1000 BG (40 mg/ml) was dissolved in growth medium, sterile filtered (0.20 μm, Corning) and immediately used. A constant dilution factor was used in each experiment for the preparation of eight test concentrations of test extracts.

In each experiment, 1×10⁴ cells per well were seeded in a 96-well plate (Costar, Corning). After a 24 hour incubation period, (cells form half-confluent monolayer) the cultures were treated with eight decimal geometric concentrations (see, Hackenberg, U. and H. Bartling. Messen and Rechnen im pharmakologischen Laboratorium mit einem speziellen Zahlensystem (WL24-System). Arch. Exp. Pharmakol., 1959, 235, 437-463), six wells per concentration, of each test extract, diluted in fresh medium. One 96-well plate per test extract was used in each experiment. After 24 hour treatment, each plate was examined under an inverted microscope to identify systematic cell seeding errors and growth characteristics of control and treated cells. Alterations in monolayer morphology were registered by a digital camera, adapted on an inverted microscope. Cytotoxicity was expressed as a concentration-dependent reduction of the uptake of the vital dye Neutral Red (see, Borenfreund, E. and J. Puerner. Toxicity determination in vitro by morphological alterations and neutral red absorption. Toxicology Letters, 24, 1985, 119-124). Optical density was measured at a wave length of 540 nm by Organon Teknika Reader 530. Relative cell viability, expressed as a percentage of the untreated negative controls, was calculated for each concentration.

Probit regression analysis (Statistic 4.3 software package) was applied to determine the concentration required to reduce cell viability to 50% (IC₅₀ values). Probit regression analysis transforms cell viability, expressed as a percentage, into standardized normally distributed values (probits) and produces a linearized model of the relationship between cell viability and the concentrations (expressed as decimal logarithms) of the test substance (see, Kaloyanova, F. (Ed.) Hygienic Toxicology. Med. i Phizk., Sofia, 1985).

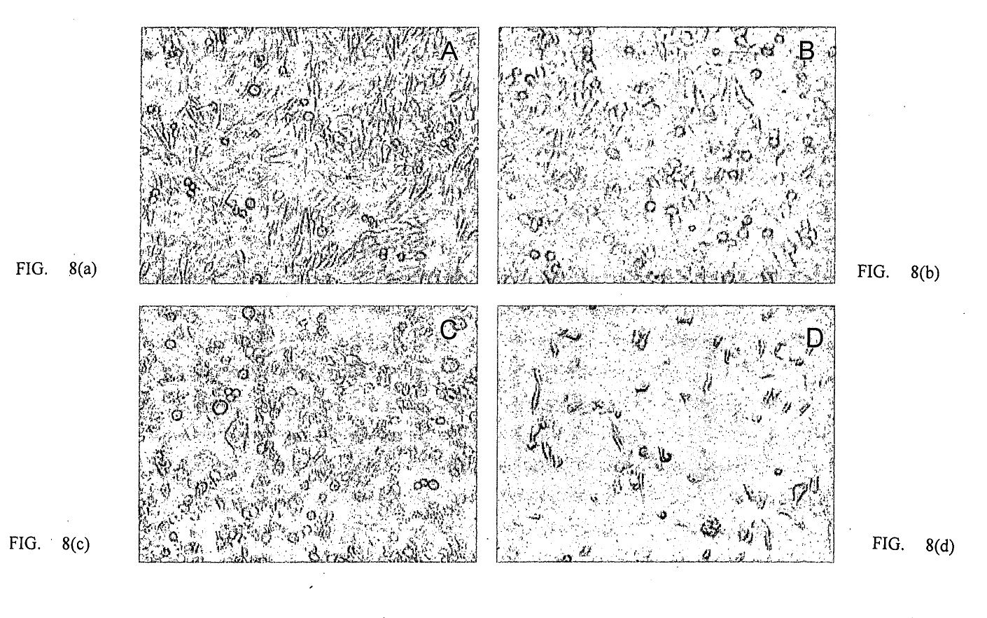

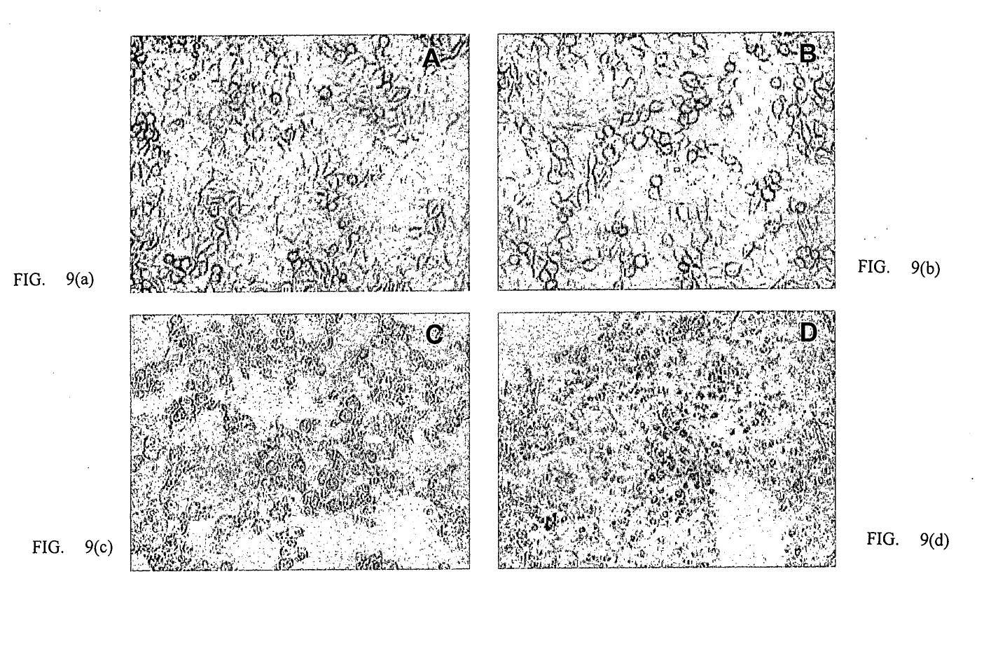

Results indicated a clear dose-dependent cytotoxic effect of Monascus composition Strain MB 1000 BG on BALB/c 3T3 and CHO cultures, as shown in FIGS. 8a-d and FIGS. 9a-d. An exposure period of 24 hours was sufficient to reduce cell viability. Progressive alterations of cellular morphology, such as rounding up, detachment and shrinkage of dead cells, as well as formation of acellular zones were observed at the effective doses. In controls, no alterations were observed. Five consecutive experiments indicated that the mean IC₅₀ for Monascus composition, Strain MB 1000 BG tested on BALB/c 3T3 standard cell line was 20 mg/ml. The mean IC₅₀ for Monascus composition Strain MB 1000 BG tested on CHO standard cell line was 30 mg/ml. The results from the experiments with each of the cell lines, obtained from normal animal embryos, indicate very low cytotoxicity of Monascus composition Strain MB 1000 BG.

### EXAMPLE 6

### Analysis of Antibacterial Activity

The study of antibacterial activity of an aqueous solution of the substance Monascus purpureus strain MB 1000 was carried out in Bulgarian Academy of Science-Institute of Microbiology in Sofia, Bulgaria by the ‘agar cup method’ described by Spooner and Sykes (1972) (Spooner, F, D. and G. Sykes. Laboratory assessment of antibacterial activity: in Methods in Microbiology, 7B, Norris, J. R., D. W. Ribbons (eds.) Academic Press: London; 1972, 216-217).

Briefly, 0.2 ml of bacterial suspension (1.0×10⁵ cfu/ml) was plated on tripticase soy agar layer into Petri dishes having a diameter of 10 cm. The samples were dissolved in double distilled water at a concentration of 10 mg/ml and 1 mg/ml, and sterilized by filtration through 0.22 μm plastic filter. After preparing the wells with a diameter of 10 mm per dish, 0.3 ml of each sample (10 mg/ml and 1 mg/ml) was dropped into each well. For pre-diffusion, the Petri dishes were placed for 2 hours at 4° C. The photodynamic effect was evaluated after exposure to day light for 90 minutes. The antibacterial activity was measured by the diameter of the inhibitory zones in the layer after 48 hours incubation at 37° C. An inhibitory zone with a diameter of less than 12 mm indicates a lack of activity. Control experiments with the solvent showed that it had no activity.

Three bacterial strains, Bacillus subtilis, Staphylcoccus aureus subsp. aureus and Escherichia coli were tested. The Bacillus subtilis (Ehrenberg, 1835, Cohn, 1872) strain 1709 was obtained from the National Bank for Industrial Microorganisms and cell cultures/NBIMCC/-Sofia, Bulgaria) was used. The strain is also deposited at CECT 356, NCIMB 8054, ATCC 6633, NCTC 10400, BUCSAV 425, IFO 3134, IFO 13720, CCM 1999, DSM 347, JCM 2499, CCRC 10447, IMET 10880, CIP 52.62, VKM B 720, NCFB 1733, LMG 8197 and has been used routinely for assay of antibiotics, sterility testing and media testing. The Staphylococcus aureus subsp. aureus (Rosenback, 1884) strain 509 was obtained from the National Bank for Industrial Microorganisms and cell cultures/NBIMCC/-Sofia, Bulgaria). This strain, deposited also at BTCC, ATCC 6538 P, FDA 209 P, CIP 53.156, NCIMB 8625, IFO 3061, NCTC 7477, NRRL B 313, LMG 8195, DSM 346, CCM 2022, CCUG 1828, CECT 240 has been generally used for assay of amikacin, cefazolin, cefadroil, doxycyclin, kanamycin, neomycin, oxacyclin, oxytetracycline, penicillin, penicillin G, tetracycline, tobramycin, and sterility testing. The Escherichia coli (Castellani and Chalmers, 1919) WF+ strain was obtained from the Collection of the Institute of Microbiology, Bulgarian Academy of Sciences.

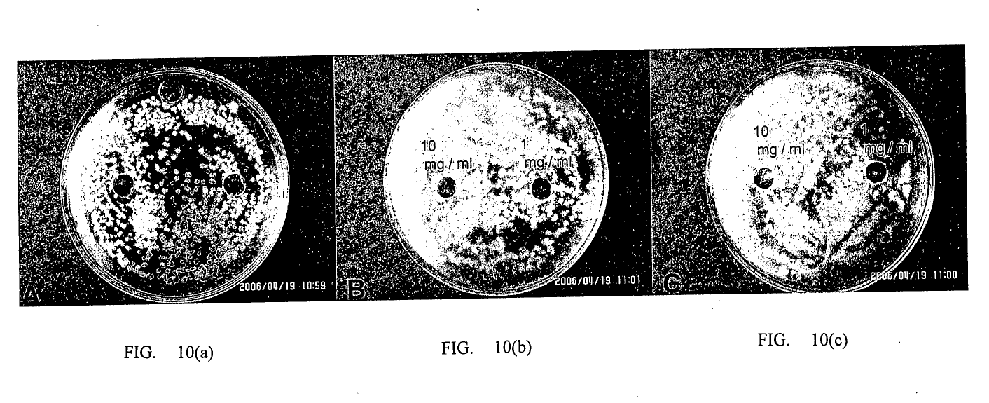

The results obtained indicate an absence of antibacterial activity of Monascus composition Strain MB 1000 BG (aqueous solution) at concentrations 1 and 10 mg/ml. FIGS. 10a-10c show control culture displaying no activity of solvent (10a), absence of antibacterial activity without daylight exposure (10b), and absence of photodynamic effect (10c) for Monascus composition Strain MB 1000 BG (aqueous solution) at concentrations 1 and 10 mg/ml on the test microorganism Bacillus subtilis, strain 1709.

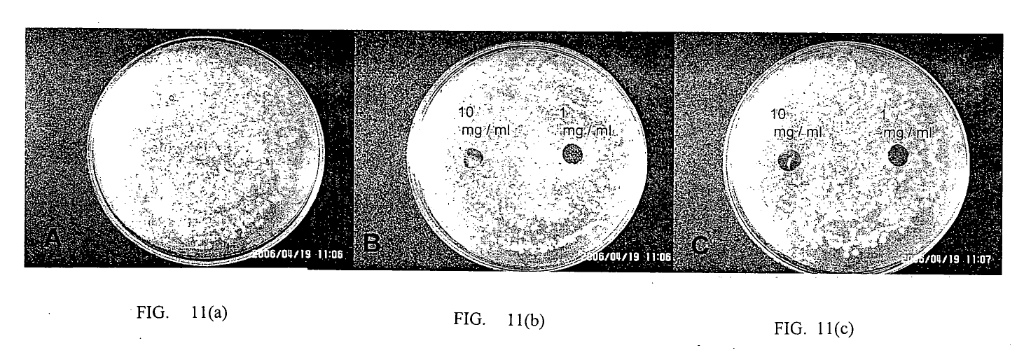

FIGS. 11a-11c show control culture (11a), absence of antibacterial activity without daylight exposure (11b), and absence of photodynamic effect (11c) for Monascus composition Strain MB 1000 BG (aqueous solution) at concentrations 1 and 10 mg/ml on the test microorganism Staphylococcus aureus subsp. aureus, strain 509.

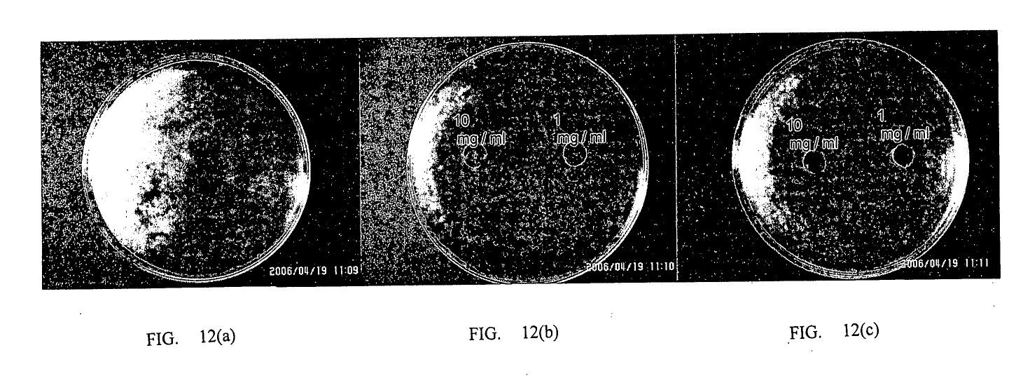

FIGS. 12a-12c show control culture displaying no activity of solvent (12a), absence of antibacterial activity without daylight exposure (12b), and absence of photodynamic effect (12c) for Monascus composition Strain MB 1000 BG (aqueous solution) at concentrations 1 and 10 mg/ml on the test microorganism Escherichia coli WF+.

The test was carried out on three different microorganisms including Staphylococcus aureus subsp. aureus Strain 509, Escherichia coli WF+ and Bacillus subtilis, strain 1709. The results obtained show the absence of antibiotic activity of Monascus composition Strain MB 1000 BG.

### EXAMPLE 7

### Analysis of Antitumor Activity

The cytotoxicities of Monascus combination, Strain MB 1000 BG on the cell cultures from the human cancer cell line MCF-7 (ductal mammary adenocarcinoma) in vitro were determined by the Neutral Red Uptake (NRU) colorimetric method and the “dye-exclusion” test.

### Cultivation

MCF-7 cells were routinely grown as monolayers in 75 cm² tissue culture flasks (Cellstar, Greiner bio-one GmbH), at 37° C. in a humidified atmosphere 5% CO₂ in RPMI 1640 medium (Sigma) supplemented with 10% foetal calf serum (BioWhittaker Europe) and antibiotics Penicillin (100 UI/ml) and Streptomycin (100 μg/ml). The cells were routinely passaged at a cell density of˜1×10⁶ cells in 75 cm² flasks every 3-4 days (average doubling time is 42-48 h).

### Test Substance

Monascus composition, Strain MB 1000 BG was used.

### Preparation of Test Substance

The test substance was dissolved in growth medium (40 mg/ml), sterile filtered (0.20 μm, Corning) and immediately used. A constant dilution factor was used in each NRU experiment for the preparation of the eight test concentrations of test extract. Four concentrations (1, 2.5, 5 and 10 mg/ml) were used in the “dye-exclusion” test.

### NRU Cytotoxicity Assay

In each experiment 1×10⁴ cells per well were seeded in 96-well plate (Costar, Corning Incorporated). After 24-h incubation period (cells form half-confluent monolayer) the cultures were treated with eight decimal geometric concentrations (Hackenberg & Bartling, 1959) (six wells per concentration) of each test extract, diluted in fresh medium. One 96-well plate per test-extract was used in each experiment. After 24-hour treatment, each plate was examined under inverted microscope to identify systematic cell seeding errors and growth characteristics of control and treated cells. Alterations in monolayer morphology were registered by an adaptor to an inverted microscope digital camera. Cytotoxicity was expressed as a concentration-dependent reduction of the uptake of the vital dye Neutral Red (Borenfreund & Puerner, 1985). Optical density was measured at wave length 540 nm by Organon Teknika Reader 530. Relative cell viability of four experiments, expressed as a percentage of the untreated negative controls, was calculated for each concentration.

### Statistical Analysis

Probit regression analysis (“Statistica 4.3” software package) was applied to determine the concentrations, required to reduce cell viability by 50% (IC₅₀ values). Probit regression analysis transforms cell viability, expressed as a percentage, into standardized normally distributed values (probits) and produces a linearized model of the relationship between cell viability and the concentrations (expressed as decimal logarithms) of the test substances (Kaloyanova, 1985).

### “Dye-exclusion” Test

The cells used in “dye-exclusion” assay were cultured in RPMI 1640 medium supplemented with 10% foetal calf serum. Cells suspended in the medium were plated in 24-well culture plates (1×10⁵ cells/ml) and incubated at 37° C. in a 5% CO₂ incubator for 24 h. The sterile test sample (10 mg/ml) was diluted in RPMI 1640 medium supplemented with 10% foetal calf serum just before use.

The solution was added to the cells in 24-well plates (two wells per each concentration; 1 ml per well) and cultured at 37° C. for 24 hours. For positive control experiments, cells were treated with 1 ml of RPMI 1640 medium (supplemented with 10% foetal calf serum) containing 5% DMSO, and the negative control cell cultures was treated with the same medium without DMSO. After the incubation period the alterations in monolayer morphology were registered by an adaptor to an inverted microscope digital camera. The cell cultures were washed with phosphate-buffered saline (PBS), trypsinized and gently suspended several times.

An aliquot of 100 μl cell suspension from each well was suspended in PBS with 2% fetal calf serum to achieve 1:10 dilution and an equal volume of Trypan Blue solution (0.4% in PBS) was added. The final cell suspension (1:20 dilution) was thoroughly mixed and the number of viable (Trypan Blue non-stained) cells was counted in a haemocytometer. There was a good reproducibility between replicate wells with standard errors below±10%. All experiments were carried out in quadruplicate. Student's t-test was used to determine the significance of the differences, compared to untreated control cell cultures.

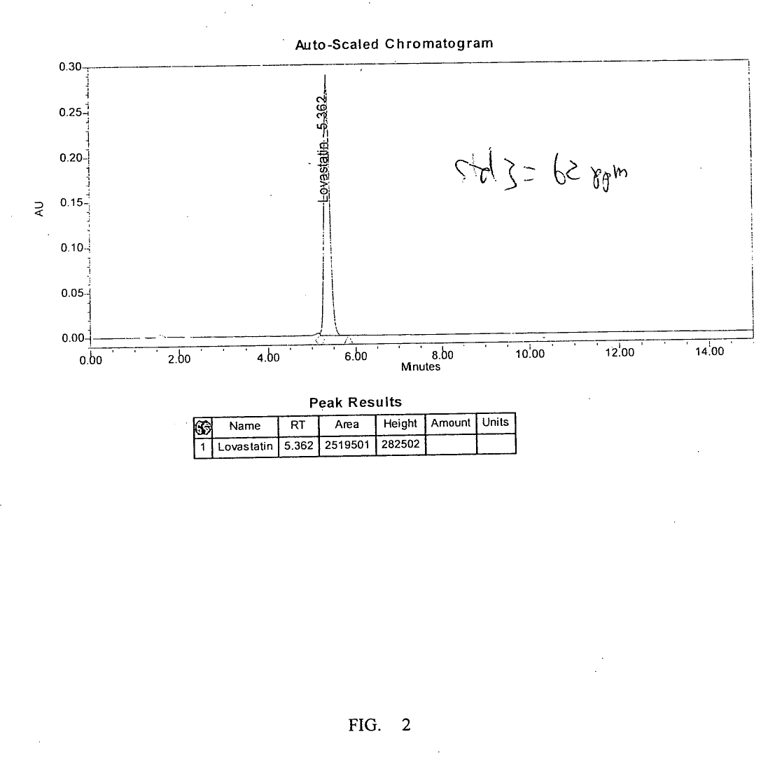

The experiments indicated a clear dose-dependent cytotoxic effect of Monascus composition, Strain MB 1000 BG on MCF-7 cultures (FIGS. 1 and 2). An exposure period of 24 hours was sufficient to reduce cell viability. Progressive alterations of cellular morphology, such as rounding-up, detachment and shrinkage of dead cells, as well as formation of acellular zones were observed at the effective doses. No alterations were observed in negative control cell cultures (FIG. 3). The results from four consecutive experiments both in NRU and “dye-exclusion” tests indicated an average IC50 value of Monascus composition, Strain MB 1000 BG, tested on MCF-7 breast carcinoma cell line of about 2.5 to about 5 mg/ml. The test substance Monascus composition, Strain MB 1000 BG, shows relatively high cytotoxicity values in MCF-7 breast carcinoma cell line after short exposure period.

The therapeutic substances disclosed herein may be administered to a patient in suitable forms (powder, liquid, tablet, capsules), and any other forms known in the art. For example, the therapeutic substances may be administered in capsule and tablet form in varying dosages, amounts and timing, depending upon the condition to be treated. A suitable treatment protocol for reduction of serum cholesterol, for example, may involve application of forms containing from about 32.5 to about 65 mg of the Monascus composition, Strain MB 1000 BG in tablets or capsules to be taken orally. The tablets or capsules may be administered at a dosage of from 2 to 3 capsules per day, taken orally from about 1 hour to about 2 hours before meals, such as for example in the morning from about 10:30 am to about 11:30 am, in the afternoon, from about 4:00 pm to about 5:30 pm and in the evening from about 8:00 pm to about 10:00 pm.

In addition, tablets or capsules comprising from about 65 mg to about 200 mg of the active substance may be administered at from 1 to 3 capsules per day from about 1 hour to about 2 hours before a meal, such as for example in the morning from about 10:30 am to about 11:30 am, in the afternoon, from about 4:00 pm to about 5:30 pm and in the evening from about 8:00 pm to about 10:00 pm. Capsules and tablets comprising the active substance may also include inactive ingredients known in the art.

In closing, it is to be understood that the embodiments described herein are illustrative of the principles of the present invention. Other modifications that may be employed are within the scope of the invention. Thus, by way of example, but not of limitation, alternative configurations may be utilized in accordance with the teachings herein. Accordingly, the drawings and description are illustrative and not meant to be a limitation thereof. While this invention has been described fully and completely with special emphasis upon preferred embodiments, it should be understood that within the scope of the appended claims the invention may be practiced otherwise than as specifically described herein.

1. A therapeutic composition for improving the circulatory system of a patient comprising the fermentate of a mixture of Monascus strains, namely Monascus composition Strain MB 100 BG, wherein said mixture comprises
from about 30.0% to about 33.33% Monascus anka,
from about 30.0% to about 33.33% Monascus kaoliang, and
from about 30.0% to about 33.33% Monascus vini.

2. The composition of claim 1, wherein the fermentate comprises no Lovastatin.

3. The composition of claim 1, wherein the fermentate is statin-free.

4. The composition of claim 1, wherein the mixture of Monascus strains has a total antioxidant activity of between about 1,200 OU/mg to about 1,800 OU/mg.

5. A statin-free therapeutic composition for lowering serum cholesterol in a patient, said composition comprising the fermentate of a mixture of Monascus anka, Monascus kaoliang, and Monascus vini.

6. The composition of claim 5, wherein the fermentate comprises no Lovastatin.

7. The composition of claim 5, wherein the fermentate is produced by the mixture of Monascus spp. after fermentation in liquid medium.

8. The composition of claim 5, wherein the fermentate has an antioxidant activity of from about 20 to about 25 times the antioxidant activity of vitamin C.

9. The composition of claim 5, wherein the fermentate has an antioxidant activity of from about 1,200 OU/mg to about 1,800 OU/mg.

10. The composition of claim 5, wherein the fermentate has an antitumor activity towards breast cancer cells.

11. A statin-free therapeutic composition for lowering serum cholesterol in a patient, said composition comprising the fermentate of a mixture of Monascus anka, Monascus kaoliang, and Monascus vini, wherein the fermentate has an antioxidant activity of from about 1,200 OU/mg to about 1,800 OU/mg and comprises no Lovastatin.

12. The composition of claim 11, wherein the fermentate has a mean LC50 in BALB/c 3T3 cells of about 20 mg/ml.

13. The composition of claim 11, wherein the fermentate has a mean LC50 in CHO cells of about 30 mg/ml.

14. The composition of claim 11, wherein the fermentate at a concentration of about 1 mg/ml displays no antibacterial activity against bacteria selected from the group consisting of Bacillus subtilis strain 1709, Staphylococcus aureus subsp. aureus strain 509 and Escherichia coli WF+.

15. The composition of claim 11, wherein the fermentate at a concentration of about 10 mg/ml displays no antibacterial activity against bacteria selected from the group consisting of Bacillus subtilis strain 1709, Staphylococcus aureus subsp. aureus strain 509 and Escherichia coli WF+.

16. The composition of claim 11, wherein the fermentate has an in vitro dose-dependent cytotoxic effect on cell cultures from the human cancer cell line MCF-7.

17. The composition of claim 16, wherein the fermentate has an average IC50 value of about between about 2.5 mg/ml to about 5 mg/ml after treating MCF-7 cells in vitro for about 24 hours.

18. A method to reduce total cholesterol levels in the serum of a patient, said method comprising the steps of:
administering a dosage of from about 32.5 to about 200 mg of a Monascus composition comprising the fermentate from a mixture of Monascus anka, Monascus kaoliang, and Monascus vini to the patient.

19. The method of claim 18, wherein the dosage is administered orally to the patient.

20. The method of claim 19, wherein the total daily dosage is about 65 mg.

21. The method of claim 18, wherein the average total reduction of total cholesterol in a patient is about 19%.

22. The method of claim 18, wherein the average total reduction of LDL-C in a patient is about 20%.
